(19) United States
(12) Patent Application Publication
(10) Pub. No.: US 2024/0246071 A1
(43) Pub. Date: Jul. 25, 2024

(54) CONTAINER FOR PROCESSING AND PRESERVING A FLUID BIOLOGICAL SAMPLE, DEVICE AND KIT RELEVANT THERETO

(71) Applicant: PEKING UNIVERSITY, BEIJING (CN)

(72) Inventors: Wei WANG, BEIJING (CN); Yaoping LIU, BEIJING (CN); Xu GAO, BEIJING (CN); Run DENG, BEIJING (CN); Tingyu LI, BEIJING (CN)

(73) Assignee: PEKING UNIVERSITY, BEIJING (CN)

(21) Appl. No.: 18/290,453

(22) PCT Filed: May 11, 2022
(86) PCT No.: PCT/EP2022/062755

§ 371 Date: Nov. 14, 2023

(30) Foreign Application Priority Data
May 14, 2021 (WO) PCT/CN2021/093842

(51) Int. Cl. B01L 3/00; G01N 1/38; G01N 35/00

(52) U.S. Cl. CPC B01L 3/502; G01N 1/38; B01L 2300/042; G01N 2035/00524

(57) ABSTRACT

A container for processing a fluid biological sample, including a container body including at least a first compartment for accommodating a first fluid and a second compartment for accommodating a second fluid, an interconnecting fluid pathway for providing a fluid communication between the first and second compartments, an inner shutter arranged so as to be movable between a first position where it blocks the interconnecting fluid pathway and a second position where it allows the fluid communication through the interconnecting fluid pathway, and a first cover arranged to allow selective access from outside the container to introduce the sample into at least one of the first and second compartments and to seal the fluid within the container body, preferably in conjunction with the inner shutter. A kit for treatment of a fluid biological sample using this container is also provided.

### CROSS-REFERENCE TO THE RELATED APPLICATIONS

This application is the national phase entry of International Application No. PCT/EP2022/062755, filed on May 11, 2022, which is based upon and claims priority to International Application No. PCT/CN2021/093842, filed on May 14, 2021, the entire contents of which are incorporated herein by reference.

### TECHNICAL FIELD

The present invention concerns a container, a device for automated processing of a fluid biological sample in conjunction with such container, and a kit for processing and preserving the fluid biological sample comprising such container and sample-treatment fluids.

### BACKGROUND

Biological samples should in general be processed promptly to allow for effective analysis and thereafter accurate diagnosis. In the case when prompt processing is not available, it is essential that the samples are pretreated or processed and then preserved properly to maintain cell viability, cell morphology etc., which are essential for detection, analysis and clinical diagnosis.

One type of fluid biological sample, for example, broncho alveolar lavage fluid (BALF) are useful for diagnosis of lung disease. Broncho alveolar lavage (BAL) is a medical procedure in which a bronchoscope is passed through the mouth or nose into the lungs, and fluid is then squirted into a small part of the lung and then recollected for analysis. The exfoliated tumor cells (ETCs) from BALF are used as clinical indicator with high sensitivity and specificity for early screening and diagnosis of lung cancer. The ETCs can be collected from the BALF by using a filter, such as the PERFECT™ (precise, efficient, rapid, flexible, easy-to-operate, controllable and thin) filter system.

However, different batches of samples or samples from different patients, can contain varied amount of target cells useful for the analysis and diagnosis, therefore modulation of the cell concentration for standardization of sample pretreatment are often desired. Besides, BALF samples, similar to other fluid biological samples, often are highly heterogeneous and mixed with various background materials such as mucus, floccules, multi-type epithelial cells, macrophages, blood cells etc. These background materials need to be processed to control its negative impact (e.g., clogging the filter, causing false positive or false negative results) to the analysis of the target cell in the biological samples. Other biological samples, such as urine, sputum, pleural effusion for clinical analysis and diagnosis may require similar processing as well before analysis. Various fluids are provided in the art for processing/pretreating the biological samples.

There are several devices in the art for processing fluid biological samples.

CN 209004058 U discloses a saliva collection and preservation tube capable of sequentially mixing saliva and multiple preservation fluids, by vertically connecting a series of tubes together. Among other deficits, the tube system in this publication requires relatively large volume sample tube, and the sequential structure is not fit for automated processing.

WO 2001/075416 A1 discloses a device for diluting and mixing a liquid sample, particularly a blood sample for performing a CRP test, leveraging capillary effect. While the device allows mixing of the sample with the treatment fluid, the capillary tube of the device is of such small volume, which limits the capacity and therefore effectiveness of processing the biological samples.

WO 2015/112496 A9 discloses a bodily fluid sample collection device comprising a cap comprising a first and second cap portions relatively movable with respect to each other to permit fluid communication between the chamber and the sample collection space. In this device the fluid communication cannot be reversed once triggered and a mixing speed cannot be controlled.

Needs exist for containers useful for processing and preserving fluid biological samples, which overcome the deficiencies in the prior art.

### SUMMARY

The present invention provides a container, preferably for processing and preserving a fluid biological sample, which provides one or more of the following advantages, such as user friendly, allow for control of the speed for proceeding the samples, is efficient and allow for standardized (such as modulating the viscosity and cell concentration of the sample) and automated sample processing.

The present invention also provides a device for automated processing of samples in conjunction with such a container.

The present invention further provides a kit for processing and preservation of a fluid biological sample, comprising said container and one or more sample-treatment fluids. The kit digests the mucus, adjusts the cell concentration and eliminates the variation caused by the negative impact of background material and cell density, and allow for more reliable parallel comparison between batches of samples (samples from different patients and samples from the same patient at different stages of disease).

In a first aspect, the invention accordingly provides a container, comprising
a container body including at least a first compartment for accommodating a first fluid and a second compartment for accommodating a second fluid;
an interconnecting fluid pathway for providing a fluid communication between the first and second compartment;
an inner shutter arranged so as to be movable between a first position where it blocks the interconnecting fluid pathway and a second position where it allows the fluid communication through the interconnecting fluid pathway;
a first cover arranged to allow selective access from outside the container to introduce the sample into at least one of the first and second compartments and to seal the fluid within the container body, preferably in conjunction with the inner shutter.

In one embodiment, the inner shutter is in the form of an inner plug and the first cover is arranged to secure the inner plug in the container body, and preferably to serve as a stopper for the movement of the inner plug at one of the first or second position.

In one embodiment, the inner shutter comprises locking means configured to releasably lock the inner shutter in the first and/or the second position.

In one embodiment, the locking means is configured to be accessed from the outside of the container to releasably unlock the inner shutter.

In one embodiment, the inner shutter is biased into either the first position or the second position by gravity or by a biasing means.

In one embodiment, the inner shutter has an engagement feature which is configured to receive a mechanical force from the outside of the container to move the inner shutter between first and second position.

In one embodiment, the engagement feature comprises a handle accessible from the outside of the container for moving the inner shutter from the first position into the second position.

In one embodiment, the container body has a second cover arranged to allow access to the first and second compartments when removed from the container body.

In one embodiment, the container body comprises a tube or cylinder including a division wall separating the first and second compartments within the volume of the tube or cylinder.

In one embodiment, a space in the tube or cylinder adjacent to an axial end portion of the division wall forms the interconnecting fluid pathway.

In one embodiment, the inner shutter in the form of the inner plug is arranged in an axial end portion of the tube or cylinder to seal the interconnecting fluid pathway by engaging with an end portion of the division wall in the first position of the inner plug.

In one embodiment, the second cover is a cap removably sealing an opposite axial end portion of the tube/cylinder.

In one embodiment, the inner shutter is arranged so as to be movable by a sliding and/or a rotating motion between the first and second positions.

In one embodiment, the container is for processing and preserving fluid biological sample, preferably broncho alveolar lavage fluid (BALF).

In one embodiment, the first fluid is a digestive fluid, and the second processing fluid is a fixative fluid and/or preservation fluid.

The digestive fluid is designed to remove the mucus and floccules for better filtration performance (clogging-free), while fixative fluid and/or preservation fluid is designed for maintaining the cell (clusters) morphology as much as possible, during a relatively long preservation period, such as a month. There are three main components in the digestive fluid, a) an enzyme or function-similar chemical to dissociate the extracellular matrix, including collagen, reticular fibers, polysaccharides, etc., b) some ionic solution to facilitate the activity of the enzyme for a sufficient digestion performance, and c) additives to maintain the integrity and morphology of cells (clusters).

Useful biological sample processing fluids are described in, for example, CN application 201810622529.5, 201610703121.1, CN202010873315.2, CN202010372894.2, CN201710128847.1.

In a second aspect, the invention also provides a device for automated processing of fluid biological samples in conjunction with the container for processing fluid biological sample according to the invention described above, comprising a chuck for holding at least one container in a defined orientation;
a drive means for moving the at least one container held in the chuck for agitating fluid in the compartments of the container;
an actuator configured to engage with the inner shutter of the at least one container held in the chuck and to move the inner shutter between its first and second position; and
a controller for operating the drive means and the actuator according to a predetermined schedule.

In one embodiment, the drive means is configured to rotate the at least one container held in the chuck around an axis thereof to agitate the fluid in the compartments of the container.

In one embodiment, the device is configured to hold plural containers for processing a sample according to the invention, while the drive means and the actuator are configured to be operated simultaneously for all containers or independently for each of the containers.

In a third aspect, the invention also provides a kit for treatment of a fluid biological sample, comprising
a container for processing a fluid biological sample according to the invention described above;
a first processing fluid accommodated in the first compartment of the container body; and
a second processing fluid accommodated in the second compartment of the container body,
wherein the inner shutter is pre-disposed in the first position where it blocks the interconnecting fluid pathway between the first and second compartments.

In one embodiment, the kit is configured for pre-treatment of a broncho alveolar lavage fluid sample (BALF sample), wherein the first processing fluid is a digestive fluid and the second processing fluid is a fixative fluid and/or preservation fluid.

The digestive fluid is designed to remove the mucus and floccules for better filtration performance (clogging-free), while the fixative fluid and/or preservation fluid is designed for maintaining the cell (clusters) morphology as much as possible, during a relatively long preservation period, such as a month. There will be mainly three components in the digestive fluid, a) an enzyme or function-similar chemical to dissociate the extracellular matrix, including collagen, reticular fibers, polysaccharides, etc., b) some ionic solution to facilitate the activity of the enzyme for a sufficient digestion performance, and c) additives to maintain the integrity and morphology of cells (clusters).

This standardized pretreatment kit for treatment of a fluid biological sample can be advantageously used to prepare a high-quality BALF sample for various related downstream detection and analysis but can be applied in applications of other fluid biological samples (e.g., urine, sputum, pleural effusion) in related clinical detection and diagnosis.

In a fourth aspect, the present invention relates to a method for automated processing of fluid biological samples, by using the device of any one of above paragraphs.

In a fifth aspect, the present invention relates to a method of processing and preserving fluid biological samples, by using the kit of any one of above paragraphs.

### BRIEF DESCRIPTION OF THE DRAWINGS

The present invention will now be described by reference to exemplary embodiments on the basis of the attached drawings:

FIG. 1 shows a schematic perspective illustration and a sectional view along a plane I-I of a container for processing fluid biological sample according to an exemplary embodiment;

FIG. 2 shows a perspective view of a device for automated processing of samples in conjunction with the container of FIG. 1;

FIG. 3 shows a schematic illustration of a workflow including stages (a) to (e) using a container of a modification of FIG. 1; and

FIGS. 4A-4F show schematic illustrations of an automated workflow including stages (a) to (f) using the container of FIG. 1 and the device of FIG. 2.

### DETAILED DESCRIPTION OF THE EMBODIMENTS

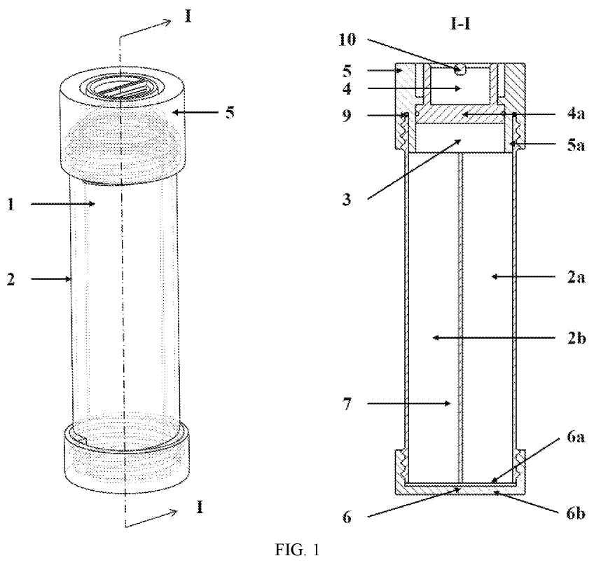

The FIG. 1 shows a schematic perspective illustration and a sectional view of a container 1 for sample processing according to an exemplary embodiment.

The container 1 has a container body (sample tube) 2 which comprises a tube or cylinder that includes at least two compartments, i.e., a first compartment 2a and a second compartment 2b, for each accommodating a fluid. The two compartments are adjacent to each other and are spaced and separated from each other within the volume of the tube/cylinder by a division wall or plate 7 to avoid a mixing of fluids unless desired as explained below.

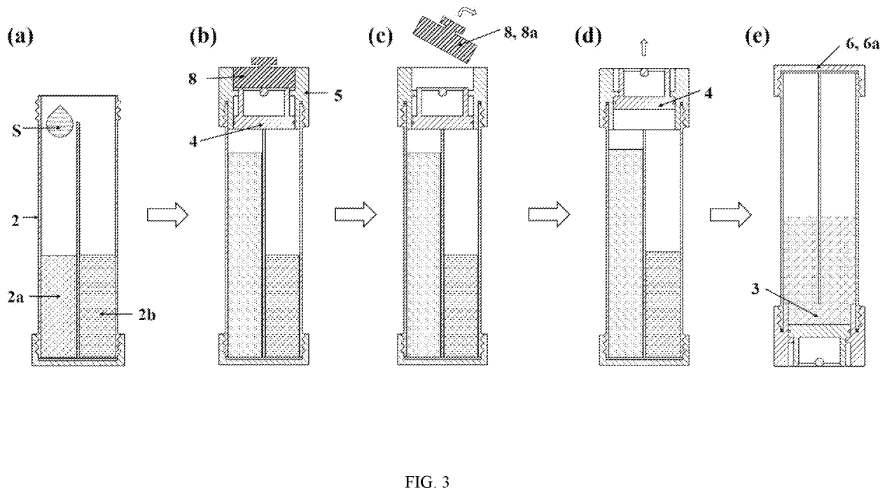

There are two sealing and functional parts on a top end and a bottom end of the container body (sample tube) 2. The sealing and functional part on the top end comprises an interconnecting fluid pathway 3 (see FIG. 3) for providing a fluid communication between the first and second compartments 2a, 2b, an inner shutter 4 arranged so as to be movable, by a sliding and/or a rotating motion, between a first position where it blocks the interconnecting fluid pathway 3 and a second position where it allows the fluid communication between the compartments 2a, 2b through the interconnecting fluid pathway 3.

The sealing and functional part on the top end also comprises a first outer cover 5 arranged to allow selective access from outside the container 1 to introduce the sample into at least one of the first and second compartments 2a, 2b and to seal the fluid with the container body 2, preferably in conjunction with the inner shutter (4) as described below.

In the embodiment of FIG. 1, the inner shutter 4 is in the form of an inner plug 4a that is arranged in an axial end portion of the container body (sample tube) 2, so as to be slidable in the longitudinal axial direction of the container body (sample tube) 2 between the first and second positions and to seal the interconnecting fluid pathway 3 that is formed in this case by a space in the container body (sample tube) 2 adjacent to an axial end portion of the division wall 7 by engaging with an axial end portion of the division wall 7 in the first position of the inner plug 4a as shown in FIG. 1. In this position an outer peripheral face of the inner plug 4a seals against an inner peripheral face of a circumferentially extending skirt 5a of the first outer cover 5 protruding downward into the inner volume of the container body (sample tube) 2. The sealing state is maintained over the length of the axial movement of the inner plug 4a within the skirt 5a between the first and second positions. Thus, the movement of the inner plug 4a controls the interconnection between the two compartments 2a, 2b. When the inner plug 4a slips down to the lower (first) position, the two compartments are disconnected, otherwise interconnected through the interconnecting fluid pathway 3. The open cross-sectional area of the interconnecting fluid pathway 3 can be scaled and depends from the position of the inner plug 4a along its movement between the first and second positions.

The first outer cover 5 is arranged to secure the inner plug 4a in the container body 2, and preferably to serve as a stopper for the sliding movement of the inner plug 4a at the second position. The first outer cover 5 is threaded to the upper end of the container body 2 and is sealed against the container body 2 by a sealing ring 9.

The inner shutter 4 or inner plug 4a preferably comprises locking means 8 configured to releasably lock the inner shutter 4 in the first and/or the second position to prevent inadvertent moving. The locking means 8 is configured to be accessed from the outside of the container 1 to releasably unlock the inner shutter 4 when desired. An example of such a locking means 8 is shown in connection with the variant of the embodiment of FIG. 3 (stages (b) and (c)). In this variant a sealing piston 8a as an example of the locking means 8 is arranged within the first outer cover 5 to fix the inner plug 4a in the first position that ensures separation of the first and second compartments 2a, 2b during transport and preservation in a situation where the fluids are pre-filled into the compartments. Before using the container to collect a sample the sealing piston 8a is to be removed from the first outer cover 5 to release the movement of the inner plug 4a.

Other types of mechanical locking means 8 like a lever, a removable locking pin, a tearable or breakable lug, a threaded engagement or a bayonet-type engagement of the inner shutter with the first outer cover are feasible and should be comprised within the disclosure.

In a variant (not shown), the inner shutter 4 is permanently biased, for example preloaded or pushed, for example by a biasing means like a spring or a resilient member and/or by gravity, into either the first position or the second position with a certain force. This feature also prevents inadvertent moving of the inner shutter until the biasing force is overcome by actively applying a counterforce or changing the orientation to allow gravity to move the inner shutter into the second position.

For the purpose of actively moving the inner shutter 4 (inner plug 4a) from the first position into the second position to initiate and control the interconnection between the two compartments 2a, 2b, the inner shutter 4 has an engagement feature 10 which is accessible from the outside of the container 1 and is configured to receive a mechanical force to move the inner shutter 4 from the first position to the second position and back, if desired or to release it such that the biasing means (if provided) will move it back into the first position. In the embodiment of FIG. 1, the engagement feature 10 comprises a physical structure like a handle or knob or protrusion or recess accessible from the outside of the container for applying an external force for moving the inner shutter 4 from the first position to the second position. The engagement feature may be designed for manual engagement or for engagement by an automated device as described later.

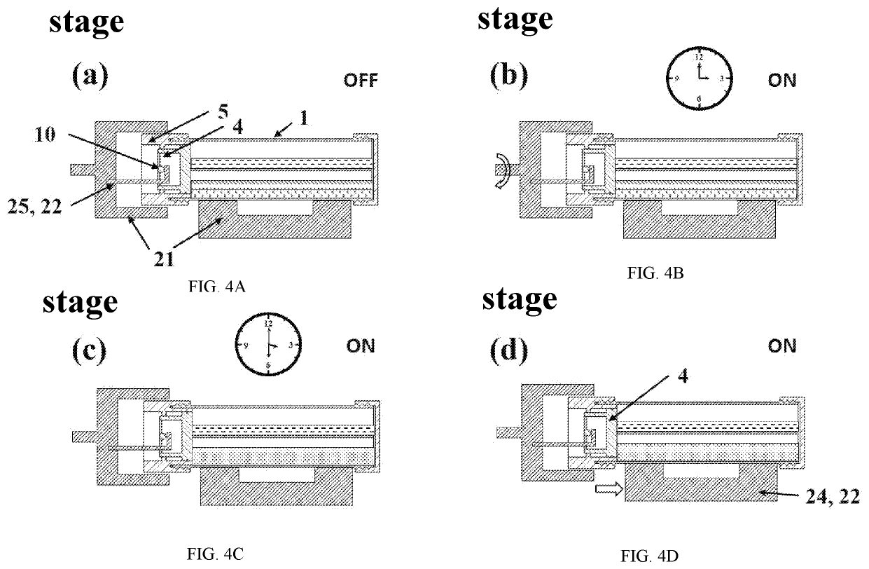

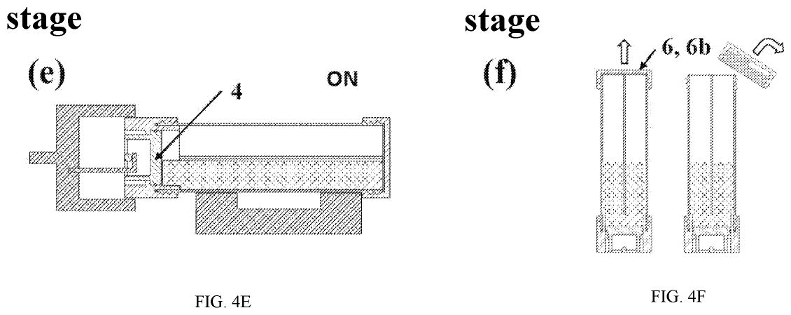

As shown in FIG. 1, the sealing of the container body 2 at the bottom axial end portion is realized by a second outer cover 6 in the form of a cap 6b removably connected with the container body 2 by a threaded engagement. Alternative connections that allow a re-attachment of the second outer cover 6 or cap 6b by a bayonet-type engagement or a spring clip or other releasable mechanical fasteners are possible. In an alternative arrangement, the second outer cover 6 or cap 6b may be attached in a manner that allows removal at a defined interface but necessarily involves destruction so that the cover/cap 6, 6b may not be re-attached. The fluid-tight sealing is achieved by an optional gasket 6a. The removal/opening of the second cover/cap 6, 6b from the container body 2 allows access to the first and second compartments 2a, 2b and sample extraction after the processing is completed (see FIG. 4F, stage (f)).

Typical dimensions of the height, outer diameter and wall thickness of the container body or sample tube 2 for the purpose of sample processing of fluid biological samples in clinical context are 110 mm, 32 mm and 1 mm, respectively, and with a volume of each compartment at 30 mL. With these dimensions the container body or sample tube 2 can be, for example fabricated from plastics material using 3D printing technology. The dimensions, in particular the wall thickness, can be smaller if molding-based manufacturing technology is used.

While two adjacent and parallel compartments are described in the embodiments above, it is possible to provide more than two such compartments in order to accommodate more than two fluids that can be selectively mixed within the container. In this case the inner shutter is preferably adapted to be movable between more than two positions in order to allow the desired fluid communication between various compartments and the desired mixing sequence as desired. To provide more than two defined interconnecting scenarios, a rotatable inner shutter with plural independently rotatable shutter portions may be adopted.

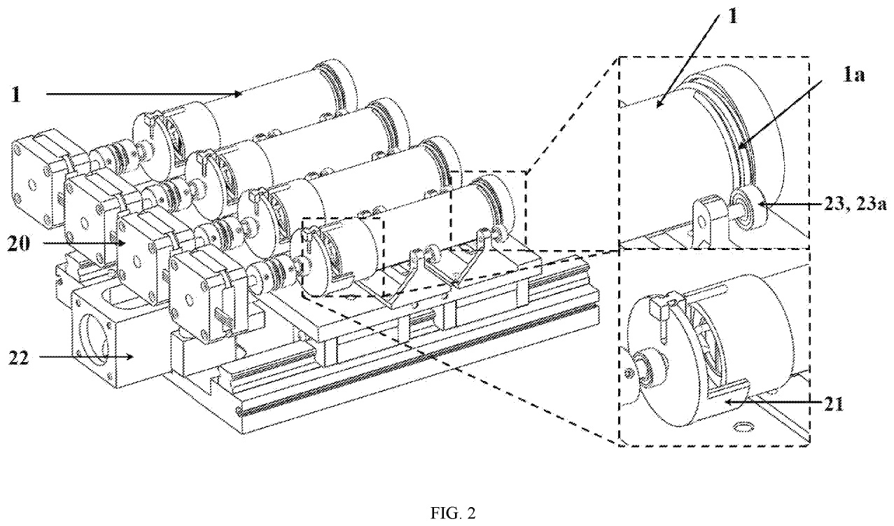

Considering that processing of fluid biological samples, for example in the clinical context, usually requires multiple sample operations at the same time, a matching device 20 for automated processing of samples in conjunction with the container 1 for processing a sample according to the invention is proposed and is shown in a schematic manner in FIG. 2.

The device 20 comprises chuck 21 for holding and locking in place at least one container 1 in a defined orientation, a drive means 23 for moving the at least one container 1 held in the chuck 21 by rotating, tilting and/or shaking it for agitating fluid in the compartments 2a, 2b of the container 1, and an actuator 22 configured to engage with the engagement feature 10 of the inner shutter 4 of the at least one container 1 held in the chuck 21 and to move the inner shutter 4 at least from its first to its second positions or between these positions.

The drive means 23 in the exemplary device 20 of FIG. 2 comprises a motor, for example a step motor which is preferably used as it allows a precise and predetermined range of motions, for rotating a friction wheel 23a engaging with an outer peripheral surface 1a of the container 1. Plural motors with friction wheels may be provided along the length of each container 1 and for each of plural chucks 21 as shown in FIG. 2 or a friction wheel may be in engagement with two or more adjacent containers at the same time. In a further variant (not shown) the drive means may alternatively comprise an endless belt trained at least partially about the circumference of one or more containers and movable by a (step) motor.

The drive means 23 in the exemplary device 20 of FIG. 2 is configured to rotate the at least one container 1 held in the chuck around an axis thereof, preferably a longitudinal center axis, to agitate the fluids in the compartments 2a, 2b of the container 1.

The actuator 22 can be preferably designed to removably engage with the engagement feature 10 on the inner shutter 4 like the handle or protrusion or recess described above. The actuator 22 may include preferably by a step motor or may include a hydraulic or pneumatic cylinder.

In the exemplary device 20 of FIG. 2, the drive means 23 for agitating the fluids in the compartments 2a, 2b of the container 1 and the actuator 22 for actuating the inner shutter 4 are implemented as separate components. It is possible, however, to combine the two functions of the drive means and of the actuator in a single component such that, for example, the actuator can actuate the inner shutter 4 and can move the container for the purpose of agitating the fluids. In the variant shown in FIGS. 4A-4F the drive means is part of the chuck engaging with the outer cover 5 for rotating the container via the outer cover.

In the chuck 21 the container body 2 of the container is supported by a platform which is movable, preferably by a step motor, under a programmable control. The required power for the movement of all the (step) motors can be satisfied, for example via an input of a low direct current voltage from an external or internal power supply (battery or other portable energy source).

The device further comprises a programmable controller for operating the drive means 23 and the actuator 22 according to a predetermined schedule demanded by the sample processing.

The device may be configured to hold plural containers 1 for processing a sample and the chuck 21 and the actuator 22 may be configured to be operated simultaneously for all containers or independently for each of the containers 1.

The working principle of the automatic device for multiple sample operation is shown in FIGS. 4A-4F. First, the container is loaded and locked onto the chuck 21 (FIG. 4A, stage (a)). In this situation an engagement device 25 that is part of the actuator 22 is engaged with the engagement feature 10 of the inner shutter 4. Second, the device is powered on and the chuck 21 starts rotation driven by a motor to rotate the container 1 for a period of time in order to sufficiently mix the sample and a pre-stored first fluid (for example a digestive fluid stored in the first compartment and a fixative fluid stored in the second compartment as described below) (FIG. 4B, stage (b)). To do so, an embedded timer is started. Over a preset period, the timer triggers the stop of chuck rotation (FIG. 4C, stage (c)) and the start of a movement of a platform 24 to pull the inner shutter 4 up (FIG. 4D, stage (d)) and get the first and second compartments interconnected (FIG. 4E, stage (e)). The digested sample is fixed after mixing with the pre-stored (fixative) fluid, and the pretreatment of the sample is finished (FIG. 4F, stage (f)) and ready for downstream processing for detection and diagnosis immediately or after a long-term storage by opening the second cover or lid 6, 6a. In this example the actuator 22 is implemented in the form of the platform 24 being movable relative to the chuck 21 holding the container and an engagement device 25 holding the inner shutter 4.

On the basis of the container for processing a fluid biological sample according to this invention in a particular application concerns a kit for standardized processing (pre-treatment) of fluid biological samples, which could eliminate the variances in terms of background materials, cell concentration etc. among clinical samples. Such a kit comprises a container 1 for processing a fluid biological sample according to the invention, a first processing fluid (for example a digestive fluid) accommodated in the first compartment 2a of the container body 2, and a second processing fluid (for example a fixative fluid) accommodated in the second compartment 2b of the container body 2. The inner shutter 4 is pre-disposed in the first position where it blocks the interconnecting fluid pathway 3 between the first and second compartments 2a, 2b and avoids premature mixing during handling and transport.

Such a kit can be in particular designed and configured for standardized pre-treatment of clinical broncho alveolar lavage fluid (BALF) samples, which could eliminate the variances among samples and fulfil non-clogging filtration in a filter system and thereby achieve a rapid and sensitive separation of the relatively rare target cells from BALF for precise diagnosis of lung cancer.

This kit includes two functional reagents including (A) a digestive fluid provided as a first fluid in the first compartment to break down and remove the mucus and (B) a fixative fluid provided as a second fluid in the second compartment to maintain the cell (cluster) morphology during long-term storage, and a container in the form of a sample tube according to the invention containing these two compartments with pre-stored aforementioned digestive and fixative fluid.

The fixative fluid is mainly for cell morphology and status maintenance, will be one single chemical or a mixture of several chemicals after tests and optimizations with clinical BALF samples.

The workflow of using the container or sample tube of FIG. 1 in the form of the aforementioned kit in a manual process is schematically shown in FIG. 3. Digestive fluid and fixative fluid are separately pre-stored in the first and second compartments 2a, 2b of the tube 2 of the sample container. First, the outer cover 5 is removed to gain access to at least the first compartment 2a which contains the digestive fluid to allow that the BALF sample S is injected into the digestive fluid compartment (FIG. 3, stage (a)). In an alternative scenario the outer cover may retain in place on the container if it has an access opening that can be opened to introduce the sample S. Then, the first outer cover 5 is again totally tightened, thereby locating the inner shutter or plug 4, 4a at the bottom position and closing the interconnecting fluid pathway (FIG. 3, stage (b)). The container is rotated or shaken for a sufficient mixing and thereby digestion (not shown in FIG. 3). Then, the locking means 8 (sealing piston 8a) is removed from the outer cover 5 (FIG. 3, stage (c)). Then, the inner shutter or plug 4, 4a is moved to the top position (FIG. 3, stage (d)) to interconnect the two compartments 2a, 2b via the interconnecting fluid pathway 3. Then, the tube is flipped or shaken or rotated to trigger the mixing of the digested BALF sample and the prestored fixative fluid (FIG. 3, stage (e)). Finally, the lower outer cover or cap 6, 6b is opened to export the pre-treated BALF sample for downstream processing.

In one embodiment this kit is completed by a matching device as described above for an automated blending of the BALF sample, digestive fluid and fixative fluid within the container in a timer interval-driven way. When a BALF sample is collected into the container, it enters into the digestive compartment. Blending of the BALF sample and digestive fluid will be performed within the compartment when the tube is loaded onto the matching device.

The sample pre-treatment procedure can be automatically fulfilled via the sample tube and matching device, with manual handling essentially only involving collecting the sample S into the first compartment of the container and loading the container onto the device after re-attaching the first or top outer cover. Over preset periods, the matching device will rotate the container to agitate its content and move the inner shutter to initiate mixing the digested BALF and the fixative fluid. Till this step, the standardized pretreatment of the BALF sample is entirely completed. The pretreated BALF sample can be stored at room temperature for at least one month with cell (cluster) state well maintained.

The use of the device avoids the dependency of heavy-duty equipment and extensive labor which exist in the present clinical detection/diagnostics, and thus expands applications in multiple sites and allows liquid biopsy of BALF-samples for lung cancer diagnosis even in remote areas.

This standardized pre-treatment kit can be used to prepare a high-quality BALF sample for various related downstream detection and analysis.

Moreover, the kit of the invention can also be expanded into applications of other fluid biological samples (e.g., urine, sputum, pleural effusion) in related clinical detection and diagnosis.

What is claimed is:

1. A container, comprising:
a container body including at least a first compartment for accommodating a first fluid and a second compartment for accommodating a second fluid;
an interconnecting fluid pathway for providing a fluid communication between the first compartment and the second compartment;
an inner shutter arranged so as to be movable between a first position where the inner shutter blocks the interconnecting fluid pathway and a second position where it the inner shutter allows the fluid communication through the interconnecting fluid pathway; and
a first cover arranged to allow selective access from an outside of the container to introduce a sample into at least one of the first compartment and the second compartment and to seal the fluid within the container body, preferably in conjunction with the inner shutter.

2. The container according to claim 1, wherein the inner shutter is in a form of an inner plug and the first cover is arranged to secure the inner plug in the container body, and preferably to serve as a stopper for the movement of the inner plug at one of the first or second position.

3. The container according to claim 1, wherein the inner shutter comprises locking means configured to releasably lock the inner shutter in the first position and/or the second position.

4. The container according to claim 3, wherein the locking means is configured to be accessed from the outside of the container to releasably unlock the inner shutter.

5. The container according to claim 1,
wherein the inner shutter is biased into either the first position or the second position by gravity or by a biasing means.

6. The container according to claim 1,
wherein the inner shutter has an engagement feature which, wherein the engagement feature is configured to receive a mechanical force from the outside of the container to move the inner shutter between the first position and the second position.

7. The container according to claim 6, wherein the engagement feature comprises a handle accessible from the outside of the container for moving the inner shutter from the first position into the second position.

8. The container according to claim 1,
wherein the container body has a second cover arranged to allow access to the first compartment and the second compartment when removed from the container body.

9. The container according to claim 1,
wherein the container body comprises a tube or cylinder including a division wall separating the first compartment and the second compartment within a volume of the tube or cylinder.

10. The container according to claim 9, wherein a space in the tube or cylinder adjacent to an axial end portion of the division wall forms the interconnecting fluid pathway.

11. The container according to claim 10, wherein the inner shutter is in a form of an inner plug and the first cover is arranged to secure the inner plug in the container body, the inner shutter in the form of the inner plug is arranged in an axial end portion of the tube or cylinder to seal the interconnecting fluid pathway by engaging with an end portion of the division wall in a first position of the inner plug.

12. The container according to claim 9, wherein the container body has a second cover arranged to allow access to the first compartment and the second compartment when removed from the container body, the second cover is a cap removably sealing an opposite axial end portion of the tube/cylinder.

13. The container according to claim 1,
wherein the inner shutter is arranged so as to be movable by a sliding and/or a rotating motion between the first position and the second position.

14. The container according to claim 1,
wherein the container is for processing and preserving a fluid biological sample, preferably broncho alveolar lavage fluid.

15. A device for automated processing of fluid biological samples in conjunction with the container according to claim 14, comprising:
a chuck for holding at least one container in a defined orientation;
a drive means for moving the at least one container held in the chuck for agitating fluid in the first compartment and the second compartment of the container;
an actuator configured to engage with the inner shutter of the at least one container held in the chuck and to move the inner shutter between the first position and the second position; and
a controller for operating the drive means and the actuator according to a predetermined schedule.

16. (canceled)

17. (canceled)

18. A kit for treatment of a fluid biological sample, comprising:
the container according to claim 14 for processing the fluid biological sample;
a first processing fluid accommodated in the first compartment of the container body; and
a second processing fluid accommodated in the second compartment of the container body,
wherein the inner shutter is pre-disposed in the first position where the inner shutter blocks the interconnecting fluid pathway between the first compartment and the second compartment.

19. (canceled)

20. A method for automated processing of fluid biological samples,
by using the device of claim 15.

21. A method of processing and preserving fluid biological samples,
by using the kit of claim 18.

22. The container according to claim 1, wherein the first cover is in conjunction with the inner shutter.

23. The container according to claim 2, wherein the first cover is arranged to serve as a stopper for a movement of the inner plug at the first position or the second position.
